(19) United States
(12) Patent Application Publication
(10) Pub. No.: US 2010/0324650 A1
(43) Pub. Date: Dec. 23, 2010

(54) HELICAL STENT GRAFT

(76) Inventors: Duncan Keeble, Oxfordshire (GB); Robert William Godbard, Oxfordshire (GB)

Correspondence Address: Intellectual Property Dept.;Dewitt Ross & Stevens SC, 2 East Mifflin Street, Suite 600, Madison, WI, 53703-2865, US

(21) Appl. No.: 12/445,682

(22) PCT Filed: Oct. 23, 2007
(86) PCT No.: PCT/GB07/04051

§ 371 Date: Jul. 21, 2009

(30) Foreign Application Priority Data
Oct. 23, 2006 (GB) 0621048.8

(51) Int. Cl. A61F 2/82

(52) U.S. Cl. 623/1.13

(57) ABSTRACT

In vivo failure of a helical stent graft is addressed by provision of reinforcing material for the graft material in at least two (preferably three) separate sections, each section comprising a continuous integral piece of material, wherein each section of reinforcing material is in the form of a helix with a longitudinal axis substantially parallel to the longitudinal axis of the tube. Thus leaks caused by unravelling of the reinforcing material are mitigated.

The present invention relates to relates to a stent graft and in particular to a helical stent graft for use in the thoracic artery.

The use of stent-grafts in minimally invasive surgery is well known in the treatment of aortic aneurysms and aneurysms of other blood vessels. Stent grafts, sometimes also known as covered stents, are also used in the gastro-intestinal tract and other tubular structures of the body where the vessel-wall has become diseased or defective in some other way.

Stent grafts are tubular structures usually constructed from two basic components: a reinforcement structure the purpose of which is to lock the stent-graft to the vessel's walls and to keep open the lumen of the vessel; and a tubular graft, which is at least partially attached to the reinforcement structure and the purpose of which is to contain the substance which would normally flow through the natural vessel.

Many designs of reinforcement structure exist; some are complex and expensive to manufacture, such as laser cut tubing, while others formed from simple wire are cheaper to manufacture.

One particular design, in which the reinforcement structure is a long helix of wire, is both economical to manufacture and has the particular benefit of being outstandingly flexible. However, there are a number of problems associated with helically reinforced stent grafts:
A first problem is that the device can be weakened significantly by a single fracture of the wire which can allow the reinforcement wire to unravel from the device. Unravelling can also be caused by slack in or damage to the stitches enabling the wire to move in the stitches and away from the sealing zones (even if the wire does not break). Either even can result in loss of radial force at the sealing zones (i.e. the ends of the stent graft) and potentially a leak.
A second problem is the limitation of using the same wire throughout the device. This prevents the selection of wires optimised to different zones of the implant.
A third is the tendency of the reinforcement loops of the helix to fall over separately, in a similar manner to a line of dominoes, resulting in the collapse of the tubular stent graft.

The present invention is intended to at least partially address the problems of helical stent grafts and is not relevant therefore to non-helical stent grafts.

In a first aspect of the present invention there is provided a stent graft comprising graft material in the form of a tube and reinforcing material for the graft material, the reinforcing material being in at least two separate sections, each section comprising a continuous integral piece of material, wherein each section of reinforcing material is in the form of a helix with a longitudinal axis substantially parallel to the longitudinal axis of the tube.

In a preferred embodiment, at least two wires, with appropriate termination, are used in sequence along the length of the stent graft. Such a construction limits the extent to which the reinforcement wire can unravel in the event of a wire break or attachment failure—for example failure of one stent section does not result in failure of the other stent section. It also permits use of wires of different properties within the same stent graft. Fixation loops may be provided at the ends of the wires to provide increased stability to the structure, reducing the risk of ‘domino’ collapse.

In a practical embodiment of a stent-graft, at least two reinforcement elements are stitched to the surface of the graft material and it is preferable that the end of one helix is attached to the graft material as close as possible to or abutting the beginning of a next helix in order to avoid a discontinuity in the reinforcement of the graft.

Alternatively, a separation can be introduced between the end of one helix and the beginning of the next so as to create a zone along the length of the stent graft with reduced stiffness. This separation may be from a sixth to a half of the diameter of the reinforcing material.

In a particularly preferred embodiment the stent graft of the present invention has three sections of reinforcing material, namely a middle section and two end sections. Preferably, the length of each end section measured in a straight line along the longitudinal axis of the stent graft is independently from 5% to 33% of the total length of the stent graft. The end sections may however be of substantially the same length.

In one embodiment, the length of each end section measured in a straight line along the longitudinal axis of the stent graft is about 20% of the total length of the stent graft.

By way of illustration, consider aortic stent grafts which have diameters which lie in the range 20 mm to 45 mm. In a preferred embodiment of the stent graft, the pitch of the helix is in the range one sixth to one half the diameter of the stent graft; in the range of practical aortic stent grafts, this would equal a pitch in the range approximately 3 mm to 23 mm. In some circumstances, it is desirable to reduce the pitch and practical devices can be made from helices with a pitch equal to the diameter of the wire itself for at least part of the overall length of the stent graft.

The ends of stent grafts are particularly critical because they provide the seal between the stent graft and the native vessel. It is preferable that at least the reinforcement components of the end seals are separate from the body of the graft so that damage to the body will not affect the critical seal zones. The minimum number of separate helices in this construction is three.

A large number of individual helices can be attached to the surface of a stent graft; in designs employing a single turn helix with a pitch of 3 mm, a graft of 200 mm overall length will employ approximately 67 separate helices.

As noted above, the seal zones at either end of a stent graft are critical areas and with the design here disclosed, wires of different stiffnesses, finishes, diameters or even materials can be used just in these critical areas. Moreover, there is merit in placing reinforcement structures within the lument of stent grafts at the seal zones to allow direct contact between the graft fabric and the native vessel. In other parts of the stent graft, it is preferable that the reinforcement wires lie on the outside of the graft so as to minimise the structures within the graft that can disrupt, for examples, the introduction of guide wires or the flow of blood.

In an alternative aspect of the invention there is provided a stent graft comprising graft material and reinforcing material for the graft material, wherein the reinforcing material is formed from a single continuous integral piece of material having at least one integral loop formed along its length for attaching the reinforcing material to the graft material.

Preferably, the reinforcing material has a plurality of integral loops along it length. It may be formed of a continuous wire formed into a helix as described above.

A usual method of attaching reinforcement wires to the surface of stent grafts is to use sewn stitches, formed either by hand or by machine. Machine stitching is usually either chain stitch or lock stitch, depending upon whether or not a second yarn is used. Both stitches are prone to ‘running’ if a yarn is broken, chain stitch being significantly more prone to this failure mode than lock stitch. Where machine stitches are used in the construction of helical and other wire stent grafts, it is preferable to stitch backwards at frequent intervals over the construction of the stent graft. Back stitching (ie stitching) over previously formed stitches) is an effective way of locking stitches in place even in the event of yarn breakage.

In a second aspect of the invention there is provided a method for implantation in the thoracic artery of a stent graft as defined above.

A preferred embodiment of the invention will now be described with reference to the drawing, in which:

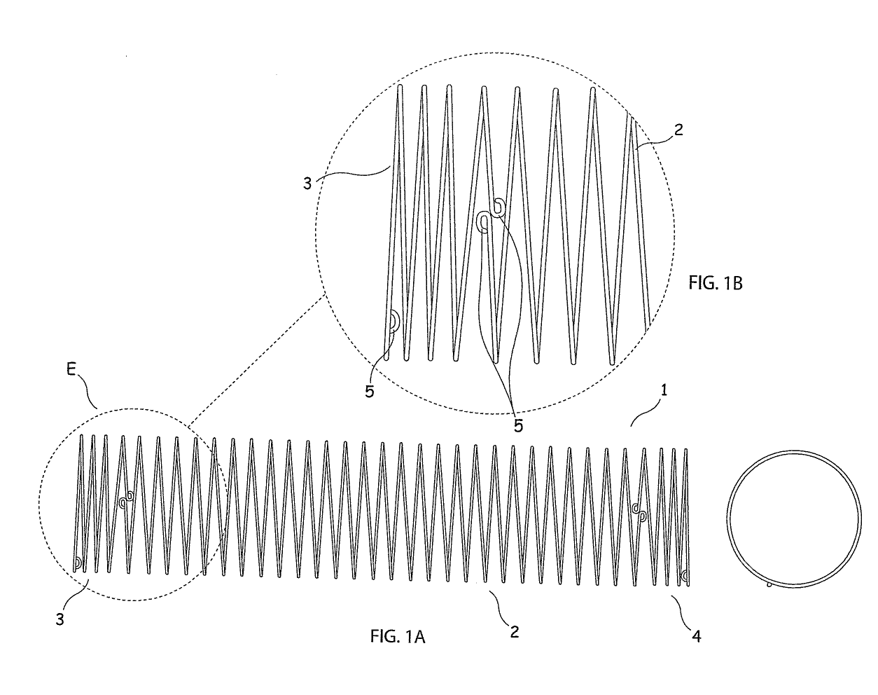

FIG. 1A depicts a thoracic stent according to the invention (with the graft material omitted for clarity);

FIG. 1B is an enlarged view of the end section E of the stent of FIG. 1A.

FIGS. 1A and 1B illustrate the fundamental construction of the reinforcement wires which form the reinforcement elements of the stent graft. Thoracic stent 1 is formed of three sections—mid-section stent 2, proximal stent 3 and distal stent 4 which are independent of each other. The fundamental shape of all three sections is a wire-form helix, usually containing at least one and usually several full turns of wire with a pitch of at least the diameter of the wire. The two free ends of each helix carry a formation or additional component intended to facilitate attachment of the free ends of the wire to the graft material so as to avoid movement of the wire or damage by the wire to the graft material. A simple formation involves constructing loop 5 at each of the ends of the wire, although alternative solutions, such as forming a series of ‘S’ bends or attaching a pre-formed fixable shape, are also effective. The loops 5 are pre-fractured to lessen the risk of unexpected fracturing occurring in use and are attached to the graft material (not shown).

In use, proximal stent 3 and distal stent 4 provide a seal with the aorta and mid-section stent 2 provides support for the graft within the aneurysm. If mid-section stent 2 fails and begins to unravel then this failure is restricted due to the separation between mid-section stent 2 and proximal stent 3 and distal stent 4.

1-11. (canceled)

12. A stent graft including:
a. graft material in the form of a tube, and
b. reinforcing material for the graft material, the reinforcing material being in first and second sections, wherein:
(1) each section includes a continuous integral piece of material in the form of a helix with a longitudinal axis substantially parallel to the longitudinal axis of the tube, and
(2) the first and second sections have respective ends which are:
(a) affixed to the graft material, and
(b) situated closely adjacent each other on the graft material,
whereby the first and second sections effectively define a continuous length of reinforcing material along the graft material.

13. The stent graft of claim 12 wherein each section of reinforcing material has opposing ends which each define a fixation loop.

14. The stent graft of claim 12 wherein the sections have at least substantially equal length measured in a straight line along the longitudinal axis of the stent graft.

15. The stent graft of claim 12 further including a third section of reinforcing material for the graft material, the third section including a continuous integral piece of material in the form of a helix with a longitudinal axis substantially parallel to the longitudinal axis of the tube, wherein the first, second, and third sections define an opposing pair of end sections with a middle section therebetween.

16. The stent graft of claim 15 wherein the length of each end section measured in a straight line along the longitudinal axis of the stent graft is from 5% to 33% of the total length of the stent graft.

17. The stent graft of claim 15 wherein the end sections are of substantially the same length measured in a straight line along the longitudinal axis of the stent graft.

18. The stent graft of claim 17 wherein the length of each end section measured in a straight line along the longitudinal axis of the stent graft is about 20% of the total length of the stent graft.

19. A stent graft including:
a. a tubular length of graft material, and
b. reinforcing material affixed along at least one of the inner and outer circumference of the graft material, wherein the reinforcing material is provided in at least two sections, each section:
(1) being formed of a continuous length of material in the form of a helix, and
(2) having opposing ends which define fixation loops, each fixation loop being affixed to the graft material.

20. The stent graft of claim 19 wherein the sections are adjacently situated along the length of graft material with their fixation loops spaced by a gap.

21. The stent graft of claim 20 wherein the gap measures from a sixth to a half of the diameter of the reinforcing material.

22. The stent graft of claim 19 wherein the sections are adjacently situated along the length of graft material with their fixation loops overlapping.

23. The stent graft of claim 19 wherein the sections are adjacently situated along the length of graft material with their fixation loops in contact with each other.

24. The stent graft of claim 23 wherein the contacting fixation loops are affixed to each other.

25. The stent graft of claim 23 wherein the contacting fixation loops are affixed to each other and to the graft material by stitching.

26. The stent graft of claim 19 wherein:
a. the tubular length of graft material extends between opposing graft ends,
b. one of the sections extends from one of the graft ends, and
c. another of the sections extends from the opposite graft end.

27. A stent graft including:
a. a tubular length of graft material extending between graft ends, and
b. sections of reinforcing material affixed along at least one of the inner and outer circumference of the graft material, wherein:
(1) each section is formed of a continuous length of material in the form of a helix, and
(2) one of the sections extends from one of the graft ends, and
(3) another of the sections extends from the other of the graft ends.

28. The stent graft of claim 27 wherein each section:
a. has opposing section ends, and
b. has at least one section end affixed to the graft material closely adjacent a section end of another of the sections.

29. The stent graft of claim 28 wherein the opposing section ends of each section define affixation loops, the affixation loops being affixed to the graft material.

30. The stent graft of claim 28 wherein adjacent section ends are spaced by a gap measuring from a sixth to a half of the diameter of the reinforcing material.

31. The stent graft of claim 28 wherein adjacent section ends are situated in contact with each other.
